(19) United States
(12) Patent Application Publication
(10) Pub. No.: US 2024/0358724 A1
(43) Pub. Date: Oct. 31, 2024

(54) BEVERAGE TABLET AND METHOD OF CUSTOMIZING AN AQUEOUS BEVERAGE USING THE SAME

(71) Applicant: North Star Brands, LLC, Apple Valley, MN (US)

(72) Inventors: Simon Cashmore, Minnetonka, MN (US); David Gonzalez-Cameron, Apple Valley, MN (US)

(21) Appl. No.: 18/648,246

(22) Filed: Apr. 26, 2024

Related U.S. Application Data
(60) Provisional application No. 63/461,908, filed on Apr. 26, 2023.

(51) Int. Cl. A61K 31/00; A23L 2/395; A23L 2/54; A23L 2/60; A61K 9/00; A61K 47/26; A61K 47/32; A61K 47/36

(52) U.S. Cl. CPC A61K 31/658; A23L 2/395; A23L 2/54; A23L 2/60; A61K 9/0095; A61K 47/26; A61K 47/32; A61K 47/36

(57) ABSTRACT

Disclosed is a beverage tablet that includes a sugar alcohol, a superdisintegrant, a binder having a solubility in 21° C. water that is less than the solubility of D-mannitol, and an active agent (e.g., THC), the tablet having a hardness of at least 1.5 kP and disintegrating in less than 10 minutes when tested according to the Water Disintegration Test Method, and a method of using the beverage tablet.

### CROSS REFERENCE TO RELATED APPLICATIONS

This application claims the benefit of U.S. Provisional Application No. 63/461,908, filed on Apr. 26, 2023, and incorporated herein.

### BACKGROUND

The invention is directed to customizing an aqueous beverage using a rapidly disintegrating tablet.

Tablets are often used to deliver pharmaceutical drugs and dietary supplements and administered by swallowing the tablet whole.

There are many different types of tablets available in the industry including, e.g., immediate-release tablets, controlled-release tablets, extended-release tablets, orally disintegrating tablets, and effervescent-release tablets. Immediate-release tablets are designed to be taken orally and dissolve in the stomach in a couple of minutes.

Controlled-release tablets are designed to be taken orally, to pass the pyloric sphincter, and to dissolve within the gastrointestinal tract based on the targeted pH.

Extended-release tablets are designed to be taken orally and release the active portion of the dose over a period of hours, either by swelling, diffusion, osmotic pump, a semi-permeable coating, or diffusion through a matrix.

Orally disintegrating tablets are designed to dissolve in the mouth in the presence of a small amount of saliva liquid that is available.

Effervescent release tablets are designed to be added to a liquid whereupon a chemical reaction occurs between an acid and a base causing the release of carbon dioxide and giving off bubbles or fizz. The aforementioned chemical reaction results in the disintegration of the tablet and the release of the active part of the tablet into the liquid. Effervescent release tablets have been adopted by the pharmaceutical industry to fit dietary supplement applications. They are typically used for immune type products and often include Vitamin C or Vitamin B. The challenge with current effervescent applications is that they are only really suitable for still (noncarbonated) and flavorless beverages such as water. The effervescent release tablet often includes a flavor agent to make the resulting beverage organoleptically appealing.

People are particular about the types of beverages they prefer to drink. At times, people want to drink a beverage with a psychoactive ingredient, but existing beverages that include a psychoactive ingredient are not to their liking (e.g., the taste or caloric content of the beverage is undesirable).

There is a need for a tablet that rapidly dissolves in a carbonated beverage without noticeably altering the taste of the beverage. There is also a need to customize a beverage with an active agent such as a psychoactive ingredient.

### SUMMARY

In one aspect, the invention features a beverage tablet that includes sugar alcohol, superdisintegrant, binder having a solubility in 21° C. water that is less than the solubility of D-mannitol, psychoactive cannabinoid, and lubricant, the tablet having a hardness of at least 1.5 kP and disintegrating in less than 10 minutes when tested according to the Tablet Disintegration Test Method.

In one embodiment, the sugar alcohol includes mannitol.

In some embodiments, the sugar alcohol includes mannitol and the binder includes inulin.

In other embodiments, the superdisintegrant includes crosslinked polyvinylpolypyrrolidone.

In another embodiment, the superdisintegrant includes crosslinked polyvinylpolypyrrolidone.

In one embodiment, the beverage tablet includes from 25% by weight to 90% by weight sugar alcohol, from 2% by weight to 50% by weight superdisintegrant, and from 5% by weight to 30% by weight of the binder.

In other embodiments, the sugar alcohol includes mannitol, the superdisintegrant includes polyvinylpolypyrrolidone, and the tablet includes from 25% by weight to 90% by weight mannitol, from 2% by weight to 50% by weight polyvinylpolypyrrolidone, and from 5% by weight to 30% by weight of the binder.

In another embodiment, the sugar alcohol includes mannitol, the superdisintegrant includes polyvinylpolypyrrolidone, the binder includes inulin, and the tablet includes from 25% by weight to 90% by weight mannitol, from 2% by weight to 50% by weight polyvinylpolypyrrolidone, and from 5% by weight to 30% by weight inulin.

In another aspect, the invention features a method of using a beverage tablet disclosed herein includes, the method including adding the tablet to an aqueous beverage, and allowing the tablet to disintegrate in the aqueous beverage.

In other aspects, the invention features a method of customizing a beverage, the method including selecting a beverage, and adding a tablet disclosed herein to the beverage.

In some embodiments, the beverage is a carbonated beverage.

In one embodiment, the beverage is a carbonated beverage having a carbonation of at least 1 volume of carbon dioxide. In another embodiment, the beverage is a carbonated beverage having a carbonation of at least 3.6 volumes of carbon dioxide.

In another aspect, the invention features a beverage tablet that includes sugar alcohol, superdisintegrant, binder having a solubility in 21° C. water that is less than the solubility of D-mannitol, an active agent, and lubricant, the tablet having a hardness of at least 1.5 kP and disintegrating in less than 10 minutes when tested according to the Water Disintegration Test Method.

The invention features a method of customizing a beverage by adding a tablet that includes an active agent and dissolves rapidly in an aqueous beverage. In some embodiments, the tablet does not impart a perceptible taste to the beverage.

Other features and advantages will be apparent from the following description of the preferred embodiments and from the claims.

### DETAILED DESCRIPTION

The tablet includes sugar alcohol, a superdisintegrant, a binder having a solubility in 21° C. water that is less than the solubility of D-mannitol, an active agent, and lubricant. A consumer can consume the active agent by adding the tablet to the beverage and allowing the tablet to disintegrate. The tablet disintegrates rapidly in a cold beverage and preferably disintegrates in cold water in no greater than 10 minutes, no greater than 5 minutes, no greater than 2 minutes, no greater than 1 minute, or even no greater than 15 seconds when tested according to the Water Disintegration Test Method. The tablet also preferably disintegrates in a carbonated beverage having a carbonate content of from 1 volume of carbon dioxide to 3.5 volumes of carbon dioxide in no greater than 10 minutes, no greater than 5 minutes, or even no greater than 4 minutes when tested according to the Low Carbonate Disintegration Test Method. The tablet also preferably disintegrates in a carbonated beverage having a carbonate content of from 3.6 volumes of carbon dioxide to 5 volumes of carbon dioxide, in no greater than 10 minutes, no greater than 6 minutes, or even no greater than 2 minutes when tested according to the High Carbonate Disintegration Test Method.

The tablet also preferably imparts no perceptible taste when added to an aqueous beverage, e.g., water, carbonated beverages (e.g., cola flavored beverages (e.g., Coke products), lemon and lime flavored beverages (e.g., Sprite products), beers), and combinations thereof.

The tablet can disintegrate in a variety of cold beverages including, e.g., water, carbonated beverages, juices (e.g., orange juice, cranberry juice, pineapple juice), sparkling flavored waters, and cocktails.

The tablet preferably has a hardness of at least 1.5 kiloponds (kP), at least 2 kP, from 2 kP to 8 kP, from 2 kP to 7 kP, from 2 kP to 5 kP, or even from 2.5 kP to 4.5 kP.

### Sugar Alcohol

Useful sugar alcohols include, e.g., mannitol, arabitol, xylitol, ribitol, sorbitol, galactitol, fucitol, iditol, inositol, volemitol, isomalt, maltitol, lactitol, maltotriitol, maltotetraitol, polyglycitol, and combinations thereof. Preferably the sugar alcohol is mannitol.

The tablet preferably includes at least 7% from 10% by weight to 90% by weight, more preferably from 50% by weight to 90% by weight, most preferably from 65% by weight to 90% by weight sugar alcohol.

### Superdisintegrant

Useful superdisintegrants include, e.g., crosslinked polyvinylpolypyrrolidone (i.e., crospovidone), crosslinked cellulose (e.g., sodium carboxy methyl cellulose), crosslinked starch (carboxymethyl starch and sodium carboxymethyl starch), crosslinked alginic acid, calcium silicate, cross-linked amylose, cross linked sodium starch glycolate, Sodium croscarmellose and combinations thereof. A preferred superdisintegrant is crospovidone.

One example of a useful commercially available superdisintegrant is available under the POLYPLASDONE XL-10 trade designation from Ashland Specialty Ingredients (Chicago, Illinois), i.e., POLYPLASDONE XL-10 crospovidone.

The tablet preferably includes from 1% by weight to 50% by weight, more preferably from 3% by weight to 45% by weight, most preferably from 6% by weight to 20% by weight superdisintegrant.

### Binder

The binder exhibits a solubility in 21° C. water that is less than the solubility of D-mannitol in 21° C. water as determined using the naked eye. Useful binders include, e.g. polysaccharides (e.g., inulin, pectin, algin, and agar), acacia, sucrose, carboxymethylcellulose sodium, polyvinylpyrolidone, starches, copovidone, hydroxypropylmethylcellulose, ethylcellulose, hydroxypropylcellulose, carboxymethyl-cellulose, gelatin, acacia, agar, alginic acid, carbomer, ceratonia, chitosan, dextrates, dextrin, glycerol dibehenate, guar gum, xantham gum, hypromellose, inulin, isomalt, magnesium aluminum silicate, maltodextrin, poloxamer, polycarbophil, polydextrose, polyethylene oxide, polymethacrylates, sodium alginate, sucrose, hydrogenated vegetable oil, microcrystalline cellulose, hydroxy propyl cellulose, poly vinyl alcohol, polyvinylpyrrolidone and mixtures thereof.

The tablet preferably includes from 5% by weight to 30% by weight, more preferably from 5% by weight to 25% by weight, most preferably from 5% by weight to 20% by weight binder.

### Active Agent

The tablet includes an active agent. Useful active agents include, e.g., psychoactive cannabinoids (e.g., tetrahydrocannabinol (THC) (e.g., delta-9-THC, delta-8-THC, and combinations thereof), dietary supplements (e.g., vitamins, minerals, fiber, fatty acids, amino acids, and combinations thereof), and combinations thereof.

The tablet preferably includes from 0.1% by weight to 30% by weight, more preferably from 0.1% by weight to 15% by weight, most preferably from 0.1% by weight to 5% by weight active agent.

### Lubricant

The tablet includes a lubricant. Useful lubricants include, e.g., magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid, sodium lauryl sulfate, coconut oil, leucine, and combinations thereof.

The tablet preferably includes from 0.3% by weight to 10% by weight, more preferably from 0.3% by weight to 5% by weight, most preferably from 0.5% by weight to 3% by weight lubricant.

### Other Ingredients

The tablet optionally includes additional ingredients including, e.g., excipients, additional binders, additional disintegrants, fluidizers, flavoring agents, additional lubricants, flow aids, anti-caking ingredients, pH adjusters, surfactants, stabilizers, colorants, sweeteners (e.g., natural sweeteners (, e.g., mono and disaccharides) and artificial sweeteners), flavor agents, and combinations thereof.

The tablet composition can be tableted to produce a variety of tablets weights including, e.g., tablets weighing from 100 mg to 4000 mg, from 250 mg to 2500 mg, from 550 mg to 1800, from 750 to 1000 mg, or even from 800 mg to 900 mg, and tablets with target weights of 550 mg, 900 mg, and 1800 mg.

The invention will now be described by way of the following examples. All parts, ratios, percents and amounts stated in the Examples are by weight unless otherwise specified.

### EXAMPLES

### Test Procedures

Test procedures used in the examples include the following. All ratios and percentages are by weight unless otherwise indicated. The procedures are conducted at room temperature (i.e., an ambient temperature of from about 20° C. to about 25° C.) unless otherwise specified.

### Water Disintegration Test Method

Water, in an amount of 500 ml, is added to a glass measuring beaker with a volume of 750 ml at a temperature of 4° C. A thermometer is used to check the temperature right before the test. A tablet is dropped into the water in the beaker. A small spatula is inserted to the half way point of the height of the liquid and is used to gently stir the liquid at a slow speed for one full rotation of the glass. This is carried out every 60 seconds. The spatula does not make contact with the tablet. The test is concluded and the time stopped when the tablet has fully disintegrated and broken apart and small particles below 500 microns (μm) are moving around freely within the liquid. The resulting time is recorded in minutes.

### Low Carbonate Disintegration Test Method

Carbonated water having from 1 volume of carbon dioxide to 3.5 volumes of carbon dioxide, in an amount of 500 ml, is added to a glass measuring beaker with a volume of 750 ml at a temperature of 4° C. A thermometer is used to check the temperature right before the test. A tablet is dropped into the carbonated water in the beaker. A small spatula is inserted to the half way point of the height of the liquid and is used to gently stir the liquid at a slow speed for one full rotation of the glass. This is carried out every 60 seconds. The spatula does not make contact with the tablet. The test is concluded and the time stopped when the tablet has fully disintegrated and broken apart and small particles below 500 microns (μm) are moving around freely within the liquid. The resulting time is recorded in minutes.

### High Carbonate Disintegration Test Method

Carbonated water having from 3.6 volumes of carbon dioxide to 5 volumes of carbon dioxide, in an amount of 500 ml, is added to a glass measuring beaker with a volume of 750 ml at a temperature of 4° C. A thermometer is used to check the temperature right before the test. A tablet is dropped into the carbonated water in the beaker. A small spatula is inserted to the half way point of the height of the liquid and is used to gently stir the liquid at a slow speed for one full rotation of the glass. This is carried out every 60 seconds. The spatula does not make contact with the tablet. The test is concluded and the time stopped when the tablet has fully disintegrated and broken apart and small particles below 500 microns (μm) are moving around freely within the liquid. The resulting time is recorded in minutes.

### Taste Perception Test Method

Two 750 ml beakers are filled with 500 ml of the liquid to be tested. A tablet is dropped into one of the beakers and the tablet is allowed to fully disintegrate. At this point the liquid is gently stirred with a spatula for 2 rotations of the glass.

The individual conducting the test will first taste the liquid in which no tablet was added to obtain a flavor reference. The individual will then taste the liquid with the disintegrated tablet and evaluate for any flavor differences. If the tastes are the same, the result is recorded as no perceptible taste difference. If the tastes differ, the results are recorded as a perceptible taste difference.

### Examples 1-6

The tablets of Examples 1-6 were prepared by spray drying a liquid composition of % by weight delta-9-tetrahydrocannabinol (THC) in an emulsion to form a THC powder. The THC powder was then dry blended with mannitol, crospovidone, inulin, sucrose, and magnesium stearate in the amounts set forth below in Table 1, in % by weight, to form a uniform blend. The uniform blend was then tableted on a Manesty Novapress tablet press to the hardness specified in Table 1. The resulting tablets weighed between 800 mg and 900 mg. The tablets were then tested according to the Water, Low Carbonate, and High Carbonate Disintegration Time Test Methods and the Taste Perception Test Method. The results are set forth in Table 1.

TABLE 1

| | 1 | 2 | 3 | 4 | 5 | 6 |
|---|---|---|---|---|---|---|
| THC | 0.29 | 0.32 | 0.30 | 0.29 | 0.29 | 0.29 |
| Mannitol | 71.22 | 39.28 | 36.90 | 71.22 | 71.22 | 71.22 |
| Sucrose | 8.7566 | 38.6349 | 12.0992 | 8.76 | 8.76 | 8.76 |
| CrosPovidone | 7.01 | 7.73 | 7.26 | 2.46 | 7.01 | 13.17 |
| Inulin | 11.68 | 12.88 | 42.35 | 11.68 | 11.68 | 11.68 |
| Magnesium Stearate | 1.05 | 1.16 | 1.09 | 1.05 | 1.05 | 1.05 |
| Total | 100 | 100 | 100 | 100 | 100 | 100 |
| Mass | | | | | | |
| Hardness (kP) | 2-3 | 2-3 | 2-3 | 2-3 | 2-3 | 2-3 |
| Disintegration Time | | | | | | |
| Water (mins) | 1 | 3 | 9 | 4 | 1 | <1 |
| Carbonated | 3 | 3 | 9 | 8 | 3 | 2.5 |
| Water (mins) | | | | | | |
| Coca-Cola (mins) | 3 | 7 | 12 | 8 | 3 | 2.5 |
| Sprite (mins) | 3 | 7 | 12 | 8 | 3 | 2.5 |
| Beer (mins) | 4 | 7 | 15 | 12 | 4 | 4 |
| Taste Perception | | | | | | |
| Water | NPTD | NPTD | NPTD | NPTD | NPTD | NPTD |
| Carbonated Water | NPTD | NPTD | NPTD | NPTD | NPTD | NPTD |
| Coke | NPTD | NPTD | NPTD | NPTD | NPTD | NPTD |
| Sprite | NPTD | NPTD | NPTD | NPTD | NPTD | NPTD |
| Beer | NPTD | NPTD | NPTD | NPTD | NPTD | NPTD |

Coke = Coca-Cola, Regular, The Coca-Cola Company, Atlanta, Georgia
Sprite = Sprite, Regular, The Coca-Cola Company, Atlanta, Georgia
Beer = Coors light, Molson Coors, Chicago Illinois
NPTD = no perceptible taste difference
Other embodiments are within the claims.

What is claimed is:

1. A beverage tablet comprising:
sugar alcohol;
superdisintegrant;
binder having a solubility in 21° C. water that is less than the solubility of D-mannitol;
psychoactive cannabinoid; and
lubricant,
the tablet having a hardness of at least 1.5 kP and disintegrating in less than 10 minutes when tested according to the Tablet Disintegration Test Method.

2. The beverage tablet of claim 1, wherein the sugar alcohol comprises mannitol.

3. The beverage tablet of claim 1, wherein the sugar alcohol comprises mannitol and the binder comprises inulin.

4. The beverage tablet of claim 1, wherein the superdisintegrant comprises crosslinked polyvinylpolypyrrolidone.

5. The beverage tablet of claim 3, wherein the superdisintegrant comprises crosslinked polyvinylpolypyrrolidone.

6. The beverage tablet of claim 1 comprising:
from 25% by weight to 90% by weight sugar alcohol;
from 2% by weight to 50% by weight superdisintegrant; and
from 5% by weight to 30% by weight of the binder.

7. The beverage tablet of claim 1, wherein the sugar alcohol comprises mannitol, the superdisintegrant comprises polyvinylpolypyrrolidone, and the tablet comprises
from 25% by weight to 90% by weight mannitol;
from 2% by weight to 50% by weight polyvinylpolypyrrolidone; and
from 5% by weight to 30% by weight of the binder.

8. The beverage tablet of claim 1, wherein the sugar alcohol comprises mannitol, the superdisintegrant comprises polyvinylpolypyrrolidone, the binder comprises inulin, and the tablet comprises
from 25% by weight to 90% by weight mannitol;
from 2% by weight to 50% by weight polyvinylpolypyrrolidone; and
from 5% by weight to 30% by weight inulin.

9. A method of using the tablet of claim 1 comprising
adding the tablet to an aqueous beverage, and
allowing the tablet to disintegrate in the aqueous beverage.

10. A method of customizing a beverage, the method comprising:
selecting a beverage; and
adding the tablet of claim 1 to the beverage.

11. The method of claim 10, wherein the beverage is a carbonated beverage.

12. The method of claim 10, wherein the beverage is a carbonated beverage having a carbonation of at least 1 volume of carbon dioxide.

13. The method of claim 10, wherein the beverage is a carbonated beverage having a carbonation of at least 3.6 volumes of carbon dioxide.

14. A beverage tablet comprising:
sugar alcohol;
superdisintegrant;
binder having a solubility in 21° C. water that is less than the solubility of D-mannitol;
an active agent; and
lubricant,
the tablet having a hardness of at least 1.5 kP and disintegrating in less than 10 minutes when tested according to the Water Disintegration Test Method.
